SEQUENCE LISTING (5 sequences)

SEQ ID NO: 1 - protein, 1114 aa, synthetic construct
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFERRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 1114 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFERRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKV

SEQ ID NO: 3 - protein, 762 aa, synthetic construct
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 762 aa, synthetic construct
VEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKV

SEQ ID NO: 5 - protein, 574 aa, synthetic construct
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFERRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

(19) United States
(12) Patent Application Publication
(10) Pub. No.: US 2026/0035678 A1
(43) Pub. Date: Feb. 5, 2026

(54) BIOLOGICAL PRODUCT, METHOD AND APPLICATION THEREOF

(71) Applicant: SHANGHAI JENOMED BIOTECH CO., LTD., Shanghai (CN)

(72) Inventor: Yuejin Huang, Vancouver (CA)

(73) Assignee: SHANGHAI JENOMED BIOTECH CO., LTD., Shanghai (CN)

(21) Appl. No.: 19/023,362

(22) Filed: Jan. 16, 2025

(30) Foreign Application Priority Data
Jul. 30, 2024 (CN) 202411025590.3

(51) Int. Cl. C12N 9/18; A61K 38/00; A61P 43/00

(52) U.S. Cl. CPC C12N 9/18; A61P 43/00; A61K 38/00; C07K 2319/01; C07K 2319/30; C07K 2319/31; C12Y 301/01008

(57) ABSTRACT

The present application provides a biological product and a method and an application thereof. The biological product is used for the preparation of a therapeutic agent for cancer treatment, and it includes at least one of the recombinant butyrylcholinesterase-albumin (rBChE-albumin) fusion protein and the recombinant butyrylcholinesterase-Fc (rBChE-Fc) fusion protein. The biological product adjusts the balance of acetylcholine and other related metabolic pathways by supplementing at least one of the exogenous rBChE-albumin fusion protein and rBChE-Fc fusion protein to treat cancer. The present invention solves the problem that the extraction and purification of BChE from human serum may not meet the huge demand for cancer treatment. The present application further provides a biological product for neutralizing and degrading nerve agents and organophosphate compounds, hydrolyzing ghrelin, and anti-wrinkle.

### CROSS-REFERENCE TO RELATED APPLICATION

This application claims the priority benefit of Chinese application serial no. 202411025590.3, filed on Jul. 30, 2024. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Jan. 14, 2025, is named 153772-US-sequencing listing and is 9,761 bytes in size.

### BACKGROUND

### Technical Field

The present invention relates to the field of biomedical technology and, more particularly, to a biological product, a method and an application thereof.

### Description of Related Art

Cholinesterases (ChE) are divided into two main groups. Those enzymes preferentially hydrolyze acetylcholine, whose enzymatic activity is BW284C51 sensitive to chemical inhibitors, are called acetylcholinesterase (AChE) or acetylcholine acetylhydrolase. AChE, also known as true, specific, pure, erythrocyte, or type I cholinesterase, is a membrane-bound glycoprotein that is present in different molecular forms in red blood cells, nerve endings, lungs, spleen, and gray matter of the brain. Those enzymes that preferentially hydrolyze other esters, such as butyrylcholine, whose enzymatic activity is sensitive to the chemical inhibitor ISO-OMPA, are known as butyrylcholinesterase (BChE, EC 3.1.1.8). BChE is also known as pseudobutyrylcholinesterase or non-specific butyrylcholinesterase. BChE is further classified based on its charge, hydrophobicity, interaction with cell membranes or extracellular structures, and subunit composition. The enzyme, also known as plasma, serotype, benzoyl, pseudotype, or type II cholinesterase, has more than 11 isoenzyme variants. BChE preferentially uses butyrylcholine and benzoylcholine as substrates for its in vitro reactions. The enzyme is found in mammalian plasma, liver, pancreas, intestinal mucosa, central nervous system white matter, smooth muscle, and heart. Human serum BChE is a globular molecular tetrameric serine esterase with a half-life of 12 days in human blood. It neutralizes and degrades nerve agents and other organophosphate compounds in the blood, preventing them from binding to AChE. BChE can also hydrolyze acetylcholine, but its other functions are largely elusive because BChE has no known specific natural substrate.

It has been observed that the functional activity of ChE is altered in various cancers, such as cancers in the nervous system, respiratory tract, digestive tract, liver, pancreas, and other cancers. AChE or BChE activity or gene structure and function may be defective in genitourinary system tumors such as the kidney, bladder, cervix, ovarian, breast, prostate, skin, soft tissue, and hematologic-related tumors. The lower the activity of AChE or BChE in the tissues of patients with hematologic malignancies, the worse the prognosis of patients. It has been suggested that the decline in ChE activity leads to the accumulation of acetylcholine in different tissues and organs, causing an imbalance of the microenvironment, which may lead to cancer development. Therefore, it is scientifically feasible to adjust the acetylcholine balance by supplementing exogenous ChE to prevent and treat cancer.

Although human serum BChE has been obtained by a large-scale purification technique, this procedure is severely limited by the volume of human plasma needed. It is unlikely that a sufficient amount of enzyme could be purified commercially by the technique. Also, there is a risk of transmission of infectious agents such as hepatitis C and HIV, etc.

Therefore, it is necessary to provide a biological product, its use, and application to avoid the above problems in the existing art.

### SUMMARY

The object of the present invention is to provide a biological product and a method and application for the preparation of a therapeutic agent for cancer treatment, and the biological product includes at least one of the rBChE-albumin fusion protein and the rBChE-Fc fusion protein to adjust the balance of acetylcholine and other related metabolic pathways to achieve the purpose of treating cancer.

Optionally, the cancers include liver cancer, small cell lung cancer, non-small cell lung cancer, pancreatic cancer, gastric cancer, esophageal cancer, breast cancer, ovarian cancer, cervical cancer, endometrial cancer, bladder cancer, colorectal cancer, prostate cancer, glioma, head and neck cancer, or hematologic tumors.

Optionally, the amino acid sequence of the rBChE-albumin fusion protein is shown in SEQ ID NO:1 or SEQ ID NO:2. The amino acid sequence of the rBChE-Fc fusion protein is shown in SEQ ID NO:3 or SEQ ID NO:4.

Optionally, the rBChE-albumin fusion protein and the rBChE-Fc fusion protein are derived from any one of the animal expression systems, viral transduction systems, plant expression systems, bacterial expression systems, CRISPR-Cas gene editing expression systems, and yeast expression systems.

Optionally, the insect expression system is an SF9 cell line.

Optionally, the mammalian expression systems comprise the following mammalian cell expression systems: CHO, BHK, dairy cow mammary gland epithelial cell MAC-T cell line, dairy cow mammary gland epithelial cell BME-UV1 cell line, HEK293.

Optionally, the animal expression systems comprise any one or more of the mammary gland bioreactor systems from mice, rats, rabbits, goats, sheep, pigs, camels, yaks, buffaloes, and dairy cows.

Optionally, the rBChE-albumin fusion protein and the rBChE-Fc fusion protein further comprise a flexible linking peptide between the two proteins, for example, Gly-Gly-Ser-Gly-Gly-Gly-Gly-Gly-Ser-Gly-Gly-Gly-Gly-Ser linking peptide, Gly-Gly-Gly-Ser linking peptide and Gly-Gly-Gly-Gly-Ser linking peptide.

Optionally, the N-terminus or C-terminus of the rBChE-albumin fusion protein and the rBChE-Fc fusion protein further comprises a tag sequence that facilitates protein purification, and the tag sequence is any one or more of His, Flag, MBP, GST, HA and Myc.

Optionally, the biological product is absorbed into the human body through transdermal absorption, including simultaneous transdermal absorption of TD-1 or other transdermal short peptides.

Optionally, the present invention also applies a biological product in neutralizing and degrading nerve agents and organophosphate poisons, hydrolyzing ghrelin, and anti-wrinkles.

### BRIEF DESCRIPTION OF THE DRAWINGS

FIG. 1 shows the relationship between the relative enzyme activity and absorbance measured by the Ellman assay in the rabbit milk-derived rBChE dilution sample of Example 1 of the invention.

FIG. 2 shows the SDS-PAGE electrophoresis and Comas bright blue staining after the purification of rabbit milk-derived rBChE in the examples of this invention.

FIG. 3 shows the denatured SDS-PAGE electrophoresis and silver staining of mouse milk dilution samples containing the rBChE-albumin fusion protein and the rBChE-Fc fusion protein in Example 2 of the invention.

FIG. 4 shows a schematic diagram of the experimental results of rabbit milk-derived rBChE on the growth inhibition of different tumor cell lines in Example 3 of the invention.

FIG. 5 shows the SDS-PAGE electrophoresis after expressing and purifying the rBChE-Fc fusion protein in CHO cells of Example 4 of the invention.

FIG. 6 shows the non-denaturing gel electrophoresis of human serum BChE, rabbit milk-derived rBChE, and CHO cell-derived rBChE-Fc fusion protein.

FIG. 7 shows a schematic diagram of the PK profiles after intravenous or intraperitoneal injection of human serum BChE, rabbit milk-derived rBChE, and CHO cell-derived rBChE-Fc fusion protein in the experimental mouse plasma of Example 4 of the invention.

FIG. 8 shows a schematic diagram of the experimental results of the growth inhibition of different tumor cell lines by CHO cell-derived rBChE-Fc fusion protein in Example 5 of the invention.

### DESCRIPTION OF THE EMBODIMENTS

### Definitions

By “butyrylcholinesterase enzyme” or “BChE enzyme” is meant a polypeptide capable of hydrolyzing acetylcholine and butyrylcholine, and whose catalytic activity is inhibited by the chemical inhibitor iso-OMPA but not by the chemical inhibitor BW 284C51. Preferred BChE enzymes to be produced by the present invention are mammalian BChE enzymes including human BChE enzymes. The term “BChE enzyme” also encompasses pharmaceutically acceptable salts of such a polypeptide.

By “recombinant butyrylcholinesterase” or “rBChE” is meant a BChE enzyme produced by a transiently transfected, stably transfected, or transgenic host cell or animal as directed by one of the expression constructs of the invention. The term “recombinant BChE” or “rBChE” also encompasses pharmaceutically acceptable salts of such a polypeptide.

By “expression construct” or “construct” is meant a nucleic acid sequence comprising a target nucleic acid sequence or sequences whose expression is desired, operably linked to sequence elements that provide for the proper transcription and translation of the target nucleic acid sequence(s) within the chosen host cells. Such sequence elements may include a promoter, a signal sequence for secretion, a polyadenylation signal, intronic sequences, insulator sequences, and other elements described in the invention. The “expression construct” or “construct” may further comprise “vector sequences”. By “vector sequences” is meant any of several nucleic acid sequences established in the art which have utility in the recombinant DNA technologies of the invention to facilitate the cloning and propagation of the expression constructs.

By “operably linked” is meant that a target nucleic acid sequence and one or more regulatory sequences (e.g., promoters, enhancers, etc) are physically linked so as to permit expression of the polypeptide encoded by the target nucleic acid sequence within a host cell.

By “signal sequence” is meant a nucleic acid sequence which, when incorporated into a nucleic acid sequence encoding a polypeptide, directs secretion of the translated polypeptide from cells which express said polypeptide. The signal sequence is preferably located at the 5′ end of the nucleic acid sequence encoding the polypeptide, such that the polypeptide sequence encoded by the signal sequence is located at the N-terminus of the translated polypeptide. By “signal peptide” is meant the peptide sequence resulting from translation of a signal sequence.

By “mammary gland-specific promoter” is meant a promoter that drives expression of a polypeptide encoded by a nucleic acid sequence to which the promoter is operably linked, where said expression occurs primarily in the mammary cells of the mammal, wherefrom the expressed polypeptide may be secreted into the milk. Preferred mammary gland-specific promoters include the β-casein promoter and the whey acidic protein (WAP) promoter.

By “host cell” is meant a cell which has been transfected with one or more expression constructs of the invention. Such host cells include mammalian cells in in vitro culture and in vivo in animals. Preferred in vitro cultured mammalian host cells include MAC-T cells, CHO cells, and BHK cells.

By “transfection” is meant the process of introducing one or more of the expression constructs of the invention into a host cell by any of the methods well established in the art, including (but not limited to) microinjection, electroporation, liposome-mediated transfection, calcium phosphate-mediated transfection, or virus-mediated transfection. A host cell into which an expression construct of the invention has been introduced by transfection is “transfected”. By “transiently transfected cell” is meant a host cell wherein the introduced expression construct is not permanently integrated into the genome of the host cell or its progeny, and therefore may be eliminated from the host cell or its progeny over time. By “stably transfected cell” is meant a host cell wherein the introduced expression construct has integrated into the genome of the host cell and its progeny.

By “transgene” is meant any segment of an expression construct of the invention which has become integrated into the genome of a transfected host cell. Host cells containing such transgenes are “transgenic.” Animals composed partially or entirely of such transgenic host cells are “transgenic animals”. Preferably, the transgenic animals are transgenic mammals (e.g., rodents or ruminants). Animals composed partially, but not entirely, of such transgenic host cells are “chimeras” or “chimeric animals”.

To make the purpose, technical solution, and advantages of the present invention more precise, the technical solution in the embodiments of the present invention will be clearly and completely described below in conjunction with the accompanying drawings of the present invention, and it is obvious that the described embodiments are part of the embodiments of the present invention, not all embodiments. Based on the embodiments in the present invention, all other embodiments obtained by a person skilled in the art without making creative labor belong to the scope of protection of the present invention. Unless otherwise defined, the technical or scientific terms used herein shall have the ordinary meaning understood by persons of general skill in the field to which the invention belongs. Words such as “including” as used herein mean that the element or object preceding the word includes the element or object listed after the word and its equivalents without excluding other elements or objects.

To perform large-scale production of a therapeutic rBChE, the invention has established an animal mammary gland bioreactor platform, from which several high-expression lines of rabbit milk-derived rBChE were screened and obtained, and the rBChE was purified. The implementation results of this invention show that the enzymatic parameters of rabbit milk-derived rBChE are the same as those of human serum BChE.

To overcome the short half-life defect of rabbit milk-derived rBChE in vivo, the invention purified the milligram-grade rBChE-Fc fusion protein in CHO cells through transit transfection. The experimental results show that the fusion protein is the same concerning the enzyme activity and other enzymatic parameters of human serum BChE and has an even longer half-life in vivo.

Some embodiments of the invention include but are not limited to liver cancer, small cell lung cancer, non-small cell lung cancer, pancreatic cancer, stomach cancer, esophageal cancer, breast cancer, ovarian cancer, cervical cancer, endometrial cancer, bladder cancer, colorectal cancer, prostate cancer, glioma, head and neck cancer or blood tumors.

In some embodiments of this invention, the rBChE-albumin fusion protein can give not only full play to the biological activity of the rBChE but also the human serum albumin, which is the most abundant protein in the blood, maintaining the body's nutrition and osmotic pressure. Its half-life is 15-19 days, which can significantly prolong the half-life of fusion proteins and also has the function of transporting drugs.

In some embodiments of the invention, the rBChE-Fc fusion protein can not only exert the biological activity of the rBChE but also have the properties of some antibodies, such as the cytotoxic effect that can cause antibody-dependent cell-mediated, the cytotoxic effect of complement-dependent, and the phagocytic effect mediated by antibody-dependent cells.

In some embodiments of this invention, the rBChE-albumin fusion protein and the rBChE-Fc fusion protein can be derived from animal expression systems, virus transduction systems, plant expression systems, bacterial expression systems, CRISPR-Cas gene editing expression systems, and yeast systems.

In some embodiments of the invention, the animal expression systems described are selected from any one of the insect expression systems and the mammalian expression systems. There are also some specific embodiments of the invention. The mammalian expression systems include the mammalian cell expression system. Some embodiments of the invention include one or more animal expression systems from the mammary gland bioreactor systems of mice, rats, rabbits, goats, sheep, pigs, camels, yaks, buffaloes, and cows.

Specifically, the above-mentioned animal mammary gland bioreactor systems use the regulatory sequence of the milk protein gene specifically expressed by the mammary gland to construct an expression vector, introducing the said expression vector into the genomes of reproductive-age non-human female animals. The vector DNA is inserted randomly, and the recombinant protein is expressed in the mammary gland and secreted into milk. Or through transit transfection in the near-term female animal's mammary gland to obtain exogenous active proteins from secreted milk.

There are also some possible embodiments of the invention. The mammalian mammary gland and plant expression systems were obtained without genetic engineering.

In some more specific embodiments of the present invention, the insect cell expression system is an SF9 cell line.

In some more specific embodiments of the present invention, the mammalian cell expression system is a Chinese hamster ovary cell line (CHO), including but not limited to CHO-K1, CHO-DG44, and CHO-S.

In some more specific embodiments of the present invention, the mammalian cell expression system is a young hamster kidney cell line (BHK).

In some more specific embodiments of the present invention, the mammalian cell expression system is a dairy cow mammary epithelial cell MAC-T cell line, a dairy cow mammary epithelial cell BME-UV1 cell line, and other dairy cows, dairy sheep and dairy rabbit primary mammary epithelial cells and mammary epithelial cell lines.

In some other more specific embodiments of the present invention, the human cell expression system is a HEK293 cell line.

Some embodiments of the present invention in which the amino acid sequence of the rBChE-albumin fusion protein is shown as SEQID NO: 1 or SEQ ID NO:2, and the amino acid sequence of the rBChE-Fc fusion protein is shown as SEQ IDNO: 3 or SEQ ID NO:4.

It should be understood that the amino acid sequences in the rBChE-albumin fusion protein are arranged sequentially. The first specific order of arrangement is the fusion of amino acid sequences such as SEQ ID NO:1 with the rBCHE and the albumin in tandem order. The second specific order is the amino acid sequences, such as SEQ ID NO:2 with the albumin and the BChE in tandem order.

Of course, the amino acid sequence in the rBChE-albumin fusion protein is not limited to the above sequential arrangements; for example, the amino acid sequence of albumin can be inserted into the amino acid sequence of BChE.

It should be understood that the amino acid sequences in the rBChE-Fc fusion protein are arranged sequentially. The first specific order of arrangement is the fusion of amino acid sequences, such as SEQ ID NO:3, with the BChE and the Fc fragment in tandem order. The second specific order is the amino acid sequences, such as SEQ ID NO:4 with the Fc fragment and the BChE in tandem order.

It should be noted that the amino acid sequence in the rBChE-Fc fusion protein is not limited to the above sequential arrangements; for example, the amino acid sequence of the Fc fragment can be inserted into the amino acid sequence of BChE.

In some embodiments of the present invention, the rBChE-albumin fusion protein and the rBChE-Fc fusion protein further comprise a flexible linking peptide between the two proteins, for example, Gly-Gly-Ser-Gly-Gly-Gly-Gly-Gly-Ser-Gly-Gly-Gly-Gly-Ser linking peptide, Gly-Gly-Gly-Ser linking peptide and Gly-Gly-Gly-Gly-Ser linking peptide.

It should be understood that in the two application scenarios of the rBChE-albumin fusion protein, a flexible linking peptide is added between the two proteins. A flexible linker peptide is added between the two proteins in the two application scenarios of the rBChE-Fc fusion protein.

In some embodiments of the present invention, the N-terminus or C-terminus of the rBChE-albumin fusion protein and the rBChE-Fc fusion protein also comprise a tag sequence that facilitates protein purification, and the tag sequence is any one or more of His, Flag, MBP, GST, HA and Myc.

Some specific embodiments of the present invention involve the biological product absorbed into the human body through transdermal absorption, including simultaneous transdermal absorption of TD-1 or other transdermal short peptides.

In some specific embodiments of the present invention, the TD-1 is composed of 11 amino acids (Ala-Cys-Ser-Ser-Ser-Pro-Ser-Lys-His-Cys-Gly) and is the first transdermal enhancing peptide discovered by phage display technology.

In some specific embodiments of the present invention, the other transdermal short peptides further comprise a membrane-penetrating polypeptide, which is composed of 22 amino acids (Cys-His-His-His-His-Arg-Lys-Arg-Lys-Arg-Arg-Gln-Arg-Arg-Arg-Arg-Arg-Arg-His-His-His).

In some embodiments of the present invention, the transdermal short peptide is prepared by artificial synthesis.

In some embodiments of the present invention, the purity of the rBChE-albumin fusion protein, the rBCHE-Fc fusion protein, and the purity of the transdermal short peptide is greater than 95%.

In some embodiments of the present invention, the time of repeated use is comprised of daily use, every other day, or use at intervals of about one week to 26 weeks to enhance its application effect.

In some embodiments of the present invention, a biological product is also provided for the application of neutralizing and degrading nerve agents and organophosphate poisons, hydrolyzing ghrelin, and anti-wrinkles.

### Example 1. Efficient Expression of rBChE in Rabbit Milk

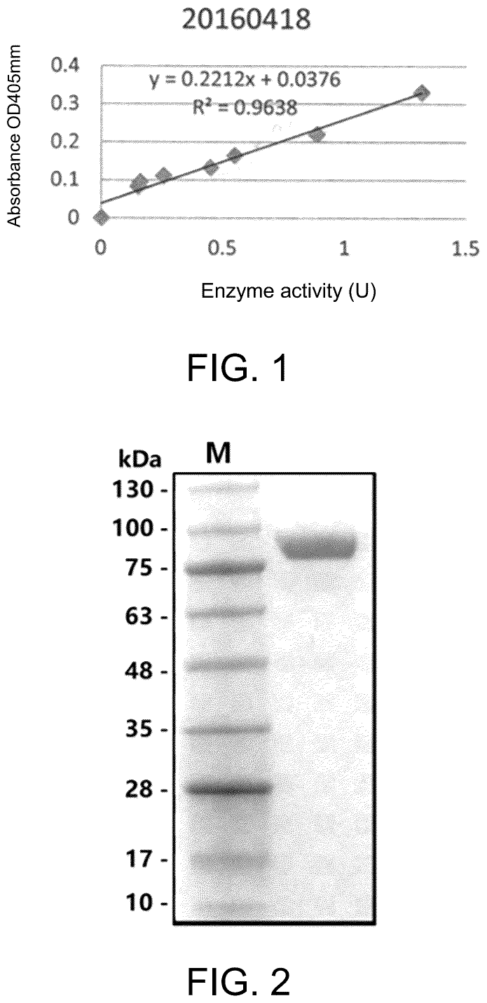

A nucleic acid plasmid encoding the rBChE protein (amino acid sequence shown in SEQ ID NO: 5) was introduced into the genome of female rabbits of reproductive age, and the rBChE was inserted randomly and expressed in the mammary gland tissue of the female rabbits, and rabbit milk was collected. The Ellman assay detected the expression concentration of rBChE in rabbit milk production. The efficient expression of rBChE in rabbit milk (0.24-12 g/L, as shown in FIG. 1 and Table 1-1) has been achieved in several positive rabbit lines, which is more than 6000 times higher than that of human serum BChE, breaking through the technical bottleneck of mass production and achieving the best technical effect unexpectedly. Rabbit milk-derived rBChE was purified by affinity and ion exchange chromatography. The washed raw rabbit milk was loaded onto a procainamide affinity column and pre-equilibrated with 10 mM phosphate buffer, pH 7.2, 1 mM EDTA, and 140 mM NaCl. Wash the column with 10-bed volumes of the same equilibration buffer and elute the protein with 10 mM phosphate buffer (pH 7.2), 1 mM EDTA, and 500 mM NaCl. The eluate was loaded onto an HQ50 ion exchange chromatography column and pre-equilibrated with the same buffer. The eluate was collected, and the column was subsequently washed with 10 mM phosphate buffer (pH 7.2), 1 mM EDTA, and 1 M NaCl to remove any trapped impurities. The purified rBChE was sterile filtered and stored at −20° C. The purified protein was tested for rBChE activity, and the total protein concentration was determined.

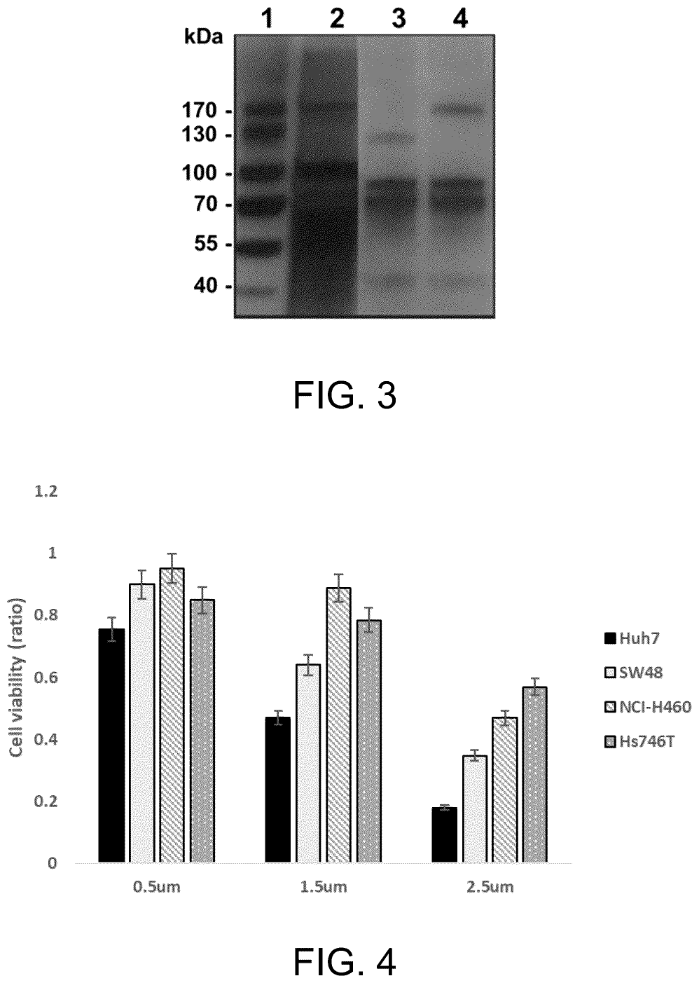

FIG. 1 shows the relationship between the relative enzyme activity and absorbance of the rabbit milk rBChE dilution sample of Example 1 of the present invention measured by the Ellman assay, and Table 1-1 is the experimental record of the rabbit milk rBChE dilution sample of Example 1 converted into concentration by the enzyme activity measured by the Ellman assay. Among them, the expression concentration of the D152 milk sample was 12 g/L. FIG. 2 shows an SDS-PAGE electrophoresis and Coomassie brilliant blue staining after purification of rBChE in Example 1 of the present invention. The molecular weight of the monomeric rBChE was approximately 95 kDa, and the purity was greater than 95%.

TABLE 1-1
Experimental record of rabbit milk-derived
rBChE dilution samples in Example 1
Experimental date: Apr. 18, 2016; Data recording date: Apr. 19, 2016.

| | | Relative enzyme | Enzyme activity | |
|---|---|---|---|---|
| Dilution | Absorption | activity (U) | in milk (g/L) * | Sample |
| 0 | 0.082 | 0 | | |
| 0 | 0 | 0 | | |
| 100 | 1.316 | 0.33 | | |
| 150 | 0.886 | 0.22 | | |
| 200 | 0.548 | 0.165 | | |
| 250 | 0.447 | 0.132 | | |
| 300 | 0.256 | 0.11 | | |
| 350 | 0.16 | 0.0946 | | |
| 400 | 0.152 | 0.0825 | | |
| 5000 | 0.612 | 0.172974 | 12.01 | D152 |
| 4000 | 0.149 | 0.070559 | 3.92 | D185 |
| 5000 | 0.11 | 0.061932 | 4.30 | D185 |
| 200 | 0.85 | 0.22562 | 0.63 | D161 |
| 200 | 0.218 | 0.085822 | 0.24 | D173 |

Experimental results: rBChE expression in D152 was at 12.01 g/L, in D185 at 3.92~4.3 g/L, in D161 at 0.63 g/L, in D173 at 0.24 g/L.

The concentrations for expression of rBChE, estimated by densitometry where 720 units=1 mg of purified BChE.

### Example 2. Efficient Expression of the rBChE-Albumin Fusion Protein and rBChE-Fc Fusion Protein in Mouse Milk

The rBChE-albumin fusion protein (as shown in SEQ ID NO: 1) and the rBChE-Fc fusion protein (as shown in SEQ ID NO: 3) protein-encoding nucleic acid plasmids were respectively introduced into the genome of female mice of reproductive age. The rBChE-albumin fusion protein and rBChE-Fc fusion protein were randomly inserted in the mouse genome. The fusion proteins were expressed in the mammary glands of the female mice of reproductive age, and mouse milk was collected. The expression concentrations of the rBChE-albumin fusion protein and rBChE-Fc fusion protein in mouse milk were detected by the Ellman assay, respectively.

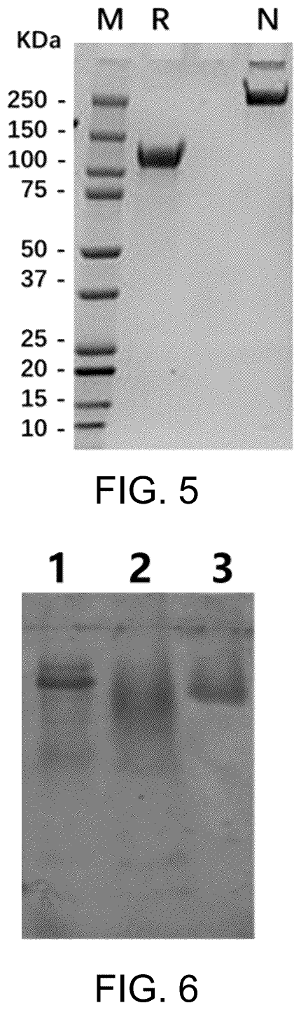

FIG. 3 shows an electropherogram of mice milk diluted samples containing rBChE-albumin fusion protein and rBChE-Fc fusion protein in Example 2 of the present invention by denaturing SDS-PAGE electrophoresis and silver staining. Among them, lane 1, protein molecular weight markers; lane 2, diluted rabbit milk sample (1:1000, with the rBChE of approximately 95 kDa as a positive control); lane 3, diluted mouse milk sample (1:1000, with the rBChE-Fc fusion protein of approximately 130 kDa); lane 4, diluted mouse milk sample (1:1000, with the rBChE-albumin fusion protein of approximately 170 kDa). The expression concentrations of the rBChE-albumin fusion protein and rBChE-Fc fusion protein in mouse milk were detected by the Ellman assay. By comparison with the lane 3 positive control sample (at 12 g/L), the expression concentration of these fusion proteins in mouse milk was estimated at approximately 1-5 g/L.

### Example 3: Growth Inhibition Experiment of Rabbit Milk-Derived rBCHE in Different Tumor Cell Lines

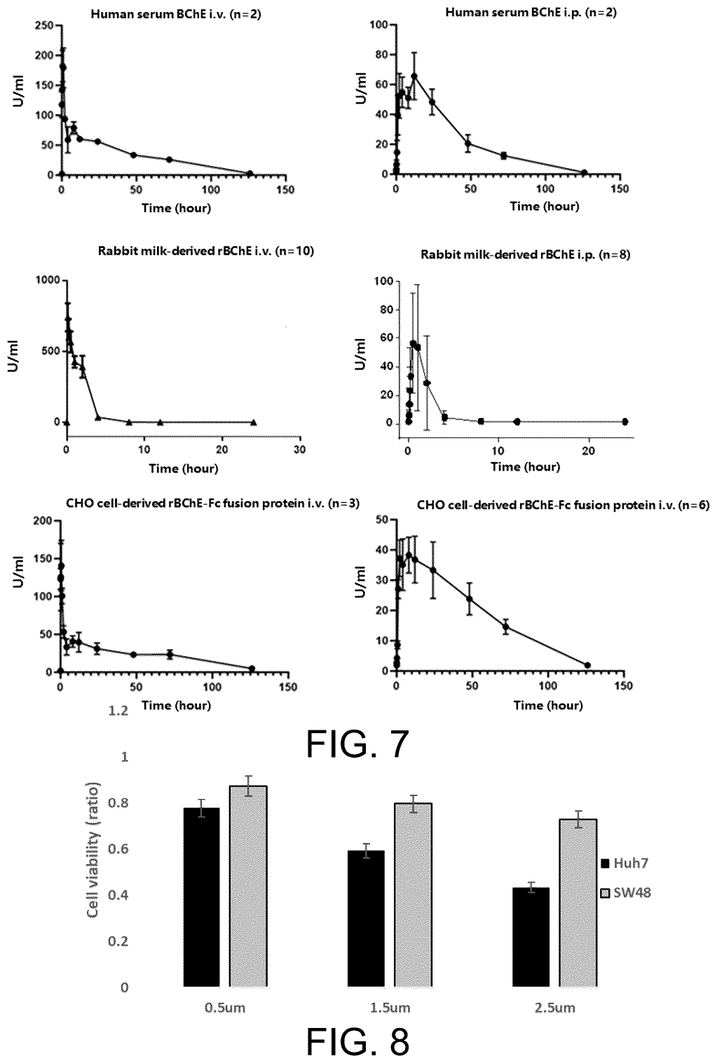

Hepatocellular carcinoma cell line Huh7 (from JCRB cell bank), colon cancer cell line SW48 (from ECACC cell bank), non-small cell lung cancer cell line NCI-H460 (from ATCC cell bank), and gastric cancer cell line Hs746T (from ATCC cell bank) were seeded in 96-well plates at 3000 cells per well in Williams' Medium E (from Sigma-Aldrich) with 10% fetal bovine serum, 5% CO₂, incubated overnight. The next day, rabbit milk-derived rBChE produced in Example 1 was added to the wells at 0.5 μm, 1.5 μm, and 2.5 μm per well. After 72 hours of incubation, 50 μl of Cell Titer Glo per well was added to the plate to detect cell proliferation. Repeated the experiments within one week. The experimental results are shown in FIG. 4; after 0.5 μm, 1.5 μm, and 2.5 μm rBChE treatment, the survival rates of Huh7 cells were 75.5%, 47%, and 18%, respectively, and the calculated half inhibitory IC50 was 1.42 μm. The survival rates of the colon cancer cell line SW48 cells were 76.5%, 54.4%, and 29.6%, respectively. The calculated IC50 was 1.63 μm, and the survival rates of NCI-H460 cells of the non-small cell lung cancer cell line NCI-H460 were 81%, 75.6%, and 39.9%, respectively, and the calculated IC50 was 2 μm; The survival rates of gastric cancer cell lines Hs746T cells were 72.3%, 66.7%, and 48.5%, respectively, and the calculated IC50 was 2.43 μm, refer to FIG. 4. The results showed that the IC50 of the rabbit milk-derived rBChE on hepatocellular carcinoma cell line Huh7 was more than 10 times lower than that of Huh7 in the commonly used hepatocellular carcinoma treatment drugs. The enzyme also had lower concentrations of the IC50 against the other cancer cell lines tested. This illustrates that the rBChE products are a potential broad-spectrum cancer therapeutic agent.

### Example 4. Expression, Purification, In Vitro Activity, and In Vivo PK Experiments of CHO Cell-Derived rBChE-Fc Fusion Protein

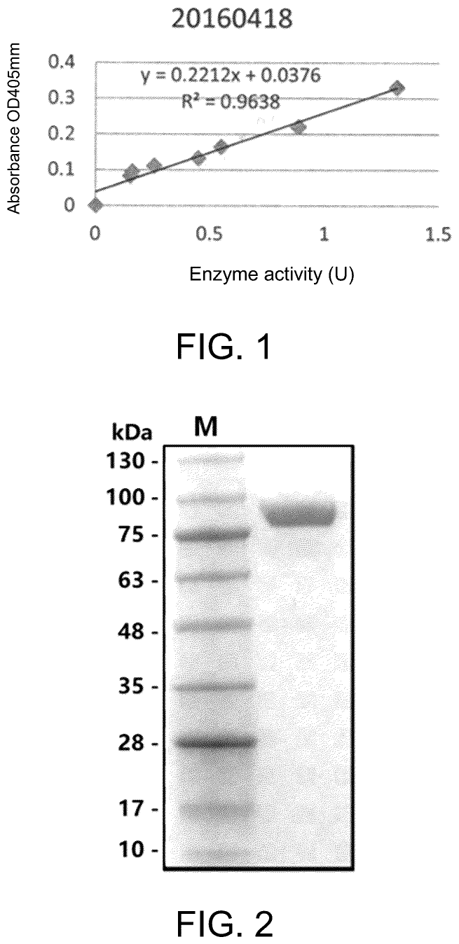

The nucleic acid plasmid encoding the rBChE-Fc fusion protein (as shown in SEQ ID NO: 4) was transiently transfected into CHO cells. The rBChE-Fc fusion protein was secreted to the cell surface, and the supernatant was collected and purified by one-step protein A column. The expression concentration of rBChE-Fc fusion protein in CHO cells was estimated at 0.15 mg/ml, the purity was 93% after one-step column purification, and the molecular weight was about 250 KDa (dimer). FIG. 5 shows the SDS-PAGE electropherogram of the rBChE-Fc fusion protein expressed and purified in CHO cells.

The enzymatic kinetic parameters and protein activities of human serum BChE, rabbit milk-derived rBChE, and CHO cell-derived rBChE-Fc fusion protein were simultaneously detected by the Ellman assay, as shown in Tables 4-1 and 4-2. The results showed that the three enzymes' enzymatic kinetic parameters and enzyme activities were similar.

TABLE 4-1
Comparison of enzymatic kinetic parameters of the three enzymes

| | Human serum | Rabbit milk- | CHO cell-derived rBChE- |
|---|---|---|---|
| Parameter | BChE | derived rBChE | Fc fusion protein |
| Vmax | 15.25 | 24.47 | 11.23 |
| Km | 20.09 | 35.44 | 15.26 |
| IC₅₀ | 3.07 × 10⁻⁵ | 2.97 × 10⁻⁵ | 2.74 × 10⁻⁵ |

TABLE 4-2
Comparison of enzyme activity of the three enzymes

| | | Protein | Specific |
|---|---|---|---|
| | Activity | concentration | activity |
| Enzyme | (U/ml) | (mg/ml) | (U/mg) |
| Human serum BChE | 15250 | 24.14 | 631.76 |
| Rabbit milk-derived rBChE | 1223.5 | 3.28 | 373.21 |
| CHO cell-derived rBChE- | 224.6 | 0.456 | 492.44 |
| Fc fusion protein | | | |

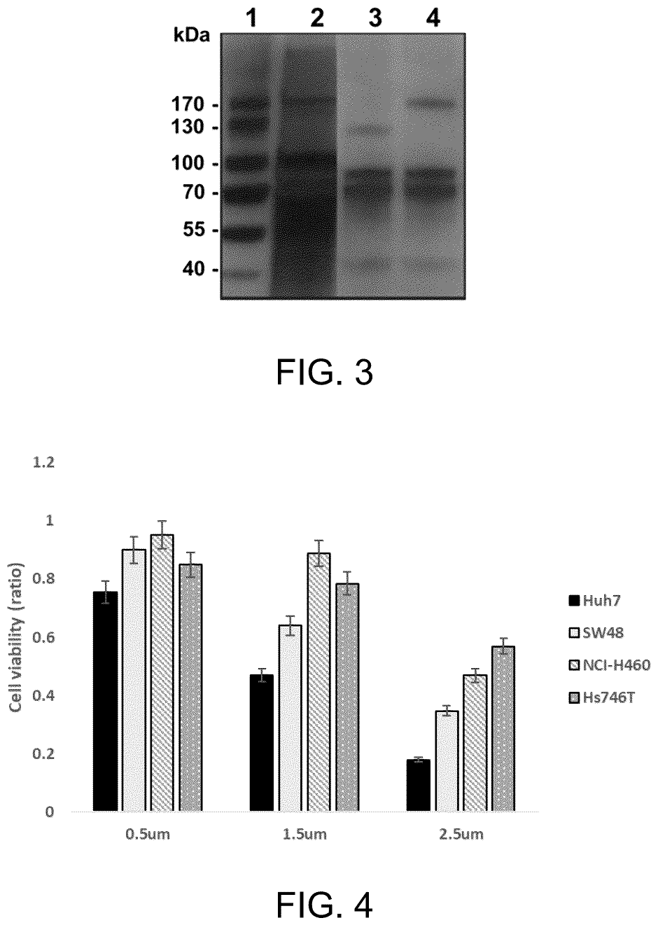

FIG. 6 shows electropherograms of human serum BChE, rabbit milk-derived rBChE, and CHO cell-derived rBChE-Fc fusion protein by native gel electrophoresis. Lane 1, the human serum BChE; lane 2, rabbit milk-derived rBChE; lane 3, CHO cells-derived rBChE-Fc fusion protein. The results showed the electrophoresis position of the rBChE-Fc fusion protein was approximately the same as that of human serum BChE.

The lyophilized preparation of human serum BChE was reconstituted with water for injection (5 mg/ml). The tail vein injection volume: body weight (g)×5 μl tail vein injection of mice (≈20 g, n=2, half male and half female). The intraperitoneal injection volume: body weight (g)×5 μl intraperitoneally injected mice (≈20 g, n=2, half male and half female). Blood was taken from the tail vein at different time points to measure enzyme activity. Blood was collected from the tail vein, and a PE tube treated with heparin was placed and left at room temperature for 30 minutes or 4° C. overnight. The supernatant was centrifuged and collected to detect the enzyme activity using the Ellman assay.

The lyophilized preparation of rabbit milk-derived rBChE was reconstituted with water for injection (5 mg/ml). The tail vein injection volume: body weight (g)×10 μl tail vein injection of mice (≈20 g, n=10, half male and half female). The intraperitoneal injection volume: body weight (g)×5 μl intraperitoneally injected mice (≈20 g, n=8, half male and half female). Blood was taken from the tail vein at different time points to measure enzyme activity. Blood was collected from the tail vein, and a PE tube treated with heparin was placed and left at room temperature for 30 minutes or 4° C. overnight. The supernatant was centrifuged and collected to detect the enzyme activity using the Ellman assay.

The tail vein injection volume of the rBChE-Fc fusion protein (1.3 mg/ml): body weight (g)×19.23 μl tail vein injection of mice (≈20 g, n=3, two females and one male). The intraperitoneal injection volume: body weight (g)×19.23 μl intraperitoneally injected mice (≈20 g, n=6, half male and half female). Blood was taken from the tail vein at different time points to measure enzyme activity. Blood was collected from the tail vein, and a PE tube treated with heparin was placed and left at room temperature for 30 minutes or 4° C. overnight. The supernatant was centrifuged and collected to detect the enzyme activity using the Ellman assay.

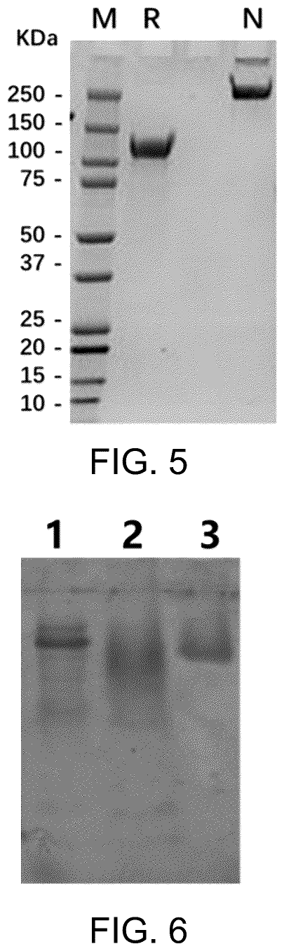

Referring to Table 4-3 and FIG. 7, the experimental results showed that the pharmacokinetic parameters of CHO cell-derived rBChE-Fc fusion protein were the same as those of human serum BCHE. Still, its half-life was 1.3 times that of human serum BChE (33.57 hours: 25.68 hours) and 35 times that of rabbit milk-derived rBChE (33.57 hours: 0.96 hours). Combined with the earlier enzymatic kinetic parameters and enzyme activity data of the present invention, it is shown that the rBChE-Fc fusion protein greatly overcomes the defect of short half-life in rabbit milk-derived rBChE without losing enzyme activity and can also be rapidly produced in CHO cells. This is a successful enzymatic molecular reconfiguration design, and its half-life in human blood is expected to reach more than 12 days.

TABLE 4-3
Comparison of pharmacokinetic parameters
of three enzymes in mice

Human serum BChE

| Parameter | 25 mg/kg (i.v.) | 25 mg/kg (i.p.) |
|---|---|---|
| T½ (h) | 25.68 ± 1.82 | 19.41 ± 1.34 |
| Tmax (h) | 0.75 ± 0.35 | 10 ± 2.83 |
| Cmax (U/mL) | 183.16 ± 27.86 | 66.41 ± 14.59 |
| AUC (U/mL × h) | 4243.60 ± 43.31 | 2886.72 ± 545.84 |
| MRT (h) | 39.55 ± 0.38 | 33.22 ± 0.0073 |
| Vz/F (L/kg) | 0.21 ± 0.014 | 0.25 ± 0.062 |
| CL(mg/(h × (U/mL))/kg) | 0.0057 | 0.0087 ± 0.0016 |

| | |
|---|---|
| Bioavailability | 68% |

Rabbit milk-derived rBChE

| Parameter | 50 mg/kg (i.v.) | 25 mg/kg (i.p.) |
|---|---|---|
| T½ (h) | 0.96 ± 0.21 | 1.53 ± 0.23 |
| Tmax (h) | 0.14 ± 0.05 | 0.79 ± 0.27 |
| Cmax (U/mL) | 753 ± 71 | 61 ± 41 |
| AUC (U/mL × h) | 1416 ± 196 | 175 ± 114 |
| MRT (h) | 1.76 ± 0.10 | 4.41 ± 1.04 |
| Vz/F (L/kg) | | |
| CL(mg/(h × (U/mL))/kg) | | |

| | |
|---|---|
| Bioavailability | 25% |

| | CHO cell-derived rBChE-Fc fusion protein | |
|---|---|---|
| Parameter | 25 mg/kg (i.v.) | 25 mg/kg (i.p.) |
| T½ (h) | 33.57 ± 4.60 | 21.45 ± 2.04 |
| Tmax (h) | 0.29 ± 0.29 | 8.8 ± 1.79 |
| Cmax (U/mL) | 139.06 ± 32.85 | 38.83 ± 6.91 |
| AUC (U/mL × h) | 3082.03 ± 139.80 | 2393.13 ± 392.20 |
| MRT (h) | 49.67 ± 3.11 | 42.04 ± 2.38 |
| Vz/F (L/kg) | 0.37 ± 0.032 | 0.32 ± 0.067 |
| CL(mg/(h × (U/mL))/kg) | 0.0075 | 0.010 ± 0.0017 |

| | |
|---|---|
| Bioavailability | 78% |

### Example 5. CHO Cells Produce rBChE-Fc Fusion Protein in Liver Cancer Cell Line Huh7 and Colon Cancer Cell Line SW48 Growth Inhibition Experiments

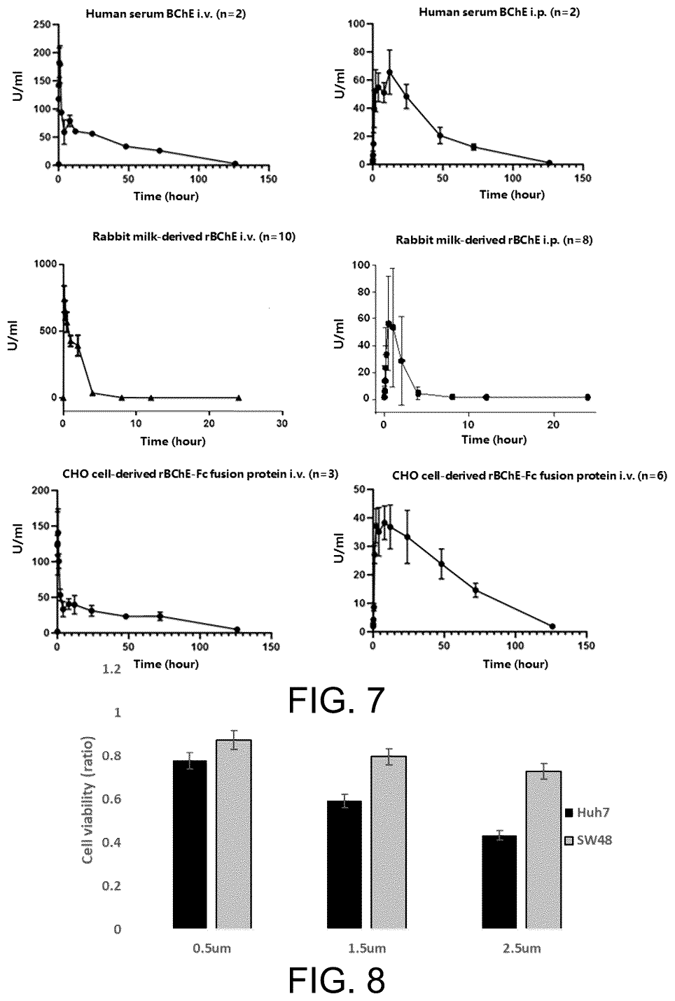

The liver cancer cell line Huh7 cells (from the JCRB cell bank) and the colon cancer cell line SW48 cells (from the ECACC cell bank) were seeded in 96-well plates at 3000 cells per well in Williams' Medium E (from Sigma-Aldrich) medium+10% fetal bovine serum, 5% CO₂, incubated overnight. The next day, the CHO cell-derived rBChE-Fc fusion protein, purified in Example 4, was added to the wells at 0.5 μm, 1.5 μm, and 2.5 μm per well. After 72 hours of incubation, 50 μl of Cell Titer Glo per well was added to the plate to detect cell proliferation. Repeated the experiments within one week. The experimental results are shown in FIG. 8. After 0.5 μm, 1.5 μm, and 2.5 μm rBChE-Fc fusion protein treatment, the survival rates of Huh7 cells were 77.9%, 59.3%, and 43.4%, respectively, and the calculated IC50 was 2.02 μm. The survival rates of the colon cancer cell line SW48 cells were 87.4%, 79.7%, and 73.1%, respectively, and the calculated IC50 was about 2.5 μm. The results showed that the growth inhibition concentration of the CHO cell-derived rBChE-Fc fusion protein on the hepatocellular carcinoma cell line Huh7 and the colon cancer cell line SW48 was similar to that of rabbit milk-derived rBChE.

Although the embodiments of the present invention have been described in detail above, it is obvious to those skilled in the art that various modifications and changes can be made to these embodiments. It should be understood, however, that such modifications and variations fall within the scope of the invention as described in the claims. Moreover, the present invention described herein may have other embodiments and be implemented or realized in various ways.

What is claimed is:

1. An application of a biological product, wherein the biological product is used to prepare a therapeutic agent for cancer treatment, and the biological product comprises at least one of rBChE-albumin fusion protein and rBChE-Fc fusion protein, and the biological product adjusts a balance of acetylcholine and other related metabolic pathways by supplementing at least one of the rBChE-albumin fusion protein and the rBCHE-Fc fusion protein to achieve a purpose of treating cancers; the cancers include liver cancer, non-small cell lung cancer, gastric cancer, and colorectal cancer; an amino acid sequence of the rBChE-albumin fusion protein is as shown in SEQ ID NO: 1 or SEQ ID NO: 2; an amino acid sequence of the rBChE-Fc fusion protein is as shown in SEQ ID NO: 3 or SEQ ID NO:4.

2. The application of the biological product according to claim 1, wherein the rBChE-albumin fusion protein and the rBChE-Fc fusion protein are derived from any one of animal expression systems, viral transduction systems, plant expression systems, bacterial expression systems, CRISPR-Cas gene editing expression systems, and yeast expression systems.

3. The application of the biological product according to claim 2, wherein the animal expression systems are selected from any one of insect expression systems and mammalian expression systems.

4. The application of the biological product according to claim 3, wherein the insect expression system comprises an SF9 cell line.

5. The application of the biological product according to claim 3, wherein the mammalian expression systems comprise following mammalian cell expression system: CHO, BHK, dairy cow mammary gland epithelial cell MAC-T cell line, dairy cow mammary gland epithelial cell BME-UV1 cell line, and HEK293.

6. The application of the biological product according to claim 2, wherein the animal expression systems comprise any one or two more from mice, rats, and rabbits.

7. The application of the biological product according to claim 2, wherein N-terminus or C-terminus of the rBChE-albumin fusion protein and the rBChE-Fc fusion protein also comprise a tag sequence that facilitates protein purification.
